(19) United States
(12) Patent Application Publication
(10) Pub. No.: US 2013/0186428 A1
(43) Pub. Date: Jul. 25, 2013

(54) Method and Apparatus for Cleaning of Laparoscopic Surgical Instruments

(71) Applicant: Midbrook, Inc., Jackson, MI (US)

(72) Inventors: Todd M. Lutz, Jackson, MI (US); John Hopkins, Rives Junction, MI (US)

(73) Assignee: MIDBROOK, INC., Jackson, MI (US)

(21) Appl. No.: 13/798,957

(22) Filed: Mar. 13, 2013

Related U.S. Application Data
(63) Continuation-in-part of application No. 12/724,622, filed on Mar. 16, 2010.
(60) Provisional application No. 61/160,384, filed on Mar. 16, 2009.

(51) Int. Cl. B08B 3/12

(52) U.S. Cl. CPC B08B 3/12
USPC 134/1; 134/184

(57) ABSTRACT

The invention is a method and apparatus for cleaning and disinfecting laparoscopic surgical instruments. The method comprises the introduction into the interior channels of the instruments of cleaning fluid heated at a predetermined temperature, and activated of the fluid by the introduction of sonic energy into the fluid at varying frequencies. The apparatus includes a unitary cleaning enclosure containing receptacles for holding the laparoscopic instruments in a bath, and for directing cleaning fluid from the bath through the interior of the instruments while providing a source of ultrasonic mechanical energy to the cleaning fluid both inside and outside the laparoscopic instruments.

### PRIORITY CLAIM

This application is a continuation-in-part of U.S. patent application Ser. No. 12/724,622, filed Mar. 16, 2010, which claims the benefit of U.S. Provisional Application No. 61/160,384, filed Mar. 16, 2009.

### FIELD OF THE INVENTION

The invention pertains to devices and methodologies for cleaning of laparoscopic surgical instruments, and more particularly, to facilitating the cleaning function utilizing a combination of heat, pressure, fluid aeration and sonic energy.

### BACKGROUND OF THE INVENTION

Laparoscopic surgery, sometimes called minimally invasive surgery, is a modern surgical technique in which operations within the human body are performed through small incisions, as compared to larger incisions utilized in traditional surgical procedures. Laparoscopic surgery commonly includes operations within the abdominal or pelvic cavities. Similar surgery performed in the thoracic or chest cavity is called thorascopic surgery. Laparoscopic and thorascopic surgery belong to a broader field of surgeries referred to as endoscopic surgeries.

These types of minimally invasive surgeries are typically performed utilizing a laparoscope. A laparoscope is a telescopic rod and lens instrument often connected to a video camera or image-sensing device. A laparoscope is also typically provided with a fiber optic system permitting the transmission of a source of illumination into the human body to illuminate the operative field. The telescopic rod and lens system and the fiber optic cable system may occupy a common instrument, or may be separate instruments to permit independent positioning of the light source and the camera during surgery. To facilitate the surgical operation, the body cavity of the patient is usually insufflated with non-reactive and non-toxic gas, such as carbon dioxide, to create an adequate working and viewing space for the surgery. In essence, the abdomen or thorax is inflated like a balloon, expanding the body wall surrounding the internal organs. Carbon dioxide is the gas of choice since it is common to the human body and is easily absorbed by tissues and easily removed by the patient's respiratory system. It is also non-flammable, which is important since electrical surgical devices are sometimes used during these procedures.

In a typical laparoscopic procedure, relatively small diameter instruments are utilized in conjunction with the laparoscope. These instruments may include graspers, scissors or clip appliers, for example. These instruments are typically inserted into the body through hollow tubes known as trocars, which provide a seal between the instrument and the tubular body of the trocar to keep the carbon dioxide from leaking from the body cavity during the procedure.

Typical laparoscopic or thorascopic working instruments are generally tubular, having a lumen or working channel, and an elongate operating actuator or plural elongate actuators positioned within the lumen. The proximal end of such a tool includes one or more actuators, while the distal end contains one or more functional device, such as graspers or scissors. This structure creates a significant potential for contamination, since body fluids and tissue may be forced into the lumen of the instrument during the surgical procedure, as a result of the pressurization of the body cavity, and the movement of the operative elements of the laparoscopic tool in relation to its tubular body. In other words, as the laparoscopic instrument is operated, a certain amount of organic material is introduced into the lumen of the tool, notwithstanding the presence of sealing elements.

It will be appreciated that the design and structure of this type of instrument presents substantial difficulties in cleaning and sterilization. It has been recognized that there are many limitations in prior art methods for cleaning this type of laparoscopic surgical instrument. This is particularly problematic in these types of applications since small particles which contaminate the interior of laparoscopic surgical instruments may harbor pathogens which, if introduced into the human body, may result in infection, illness or death.

Traditionally, a number of devices and methodologies have been attempted to effect thorough cleaning of these types of apparatus. Included among these are a variety of ultrasonic cleaning techniques, such as that disclosed in U.S. Pat. No. 3,957,552 and U.S. Pat. No. 5,380,369. More recently, methods for ultrasonically cleaning the interior of laparoscopic tools using an internal flexible wire resonator has been disclosed in U.S. Pat. No. 5,830,127.

These methodologies have, however, proved to be only partially effective in achieving absolute cleanliness of the laparoscopic surgical tool. The present invention is a simpler, yet more efficient method and apparatus for cleaning the interior channels of an elongated tubular instrument, such as laparoscopic instruments.

It is therefore an object of the present invention to provide an improved method and apparatus for cleaning and disinfecting the interior channels of elongated tubular medical instruments, such as laparoscopic instruments, and for cleaning and disinfecting the exterior of said instruments.

It is a further object to provide a method and apparatus for the generation of varying frequency ultrasonic waves in aerated fluids to effectuate cleaning within the internal cavities of elongate tubular surgical instruments.

### SUMMARY OF THE INVENTION

The present invention provides a method and apparatus for cleaning of the interior spaces and exterior surfaces of a laparoscopic or thorascopic tubular instrument. The method and process of the invention comprise the introduction into the interior channels of the instrument of a cleaning fluid, heated to a predetermined temperature, and infused with gas bubbles, followed by the introduction of sonic energy into the fluid at wave frequencies which are varied, while immersing the instrument in a bath of said cleaning fluid.

The apparatus of the present invention comprises a unitary cleaning station containing all of the elements required for completing a cleaning and disinfecting operation, and designed to be portable and easily connected and disconnected from necessary sources of electrical power, compressed air and water. The apparatus is substantially self-contained, requiring only such sources of electricity, water and air as may be conventionally found in an institutional setting in which the device will be useful.

The cleaning apparatus includes a lower enclosure portion, which houses, mounts and protects various operating elements of the system, including valves, pumps, electronic controls, actuators and transducers. Mounted above the lower enclosure is a tub designed to accept a volume of cleaning fluid, into which is immersed a plurality of trays designed to hold laparoscopic instruments to be cleaned and disinfected. A pneumatic actuator is provided to raise and lower the trays from and into a cleaning solution, and to agitate the trays to enhance the cleaning operation.

Cleaning fluid is prepared automatically by the apparatus and passed into the interior of the tub, as well as into a manifold which distributes cleaning fluid to the interior of surgical instruments to be cleaned. An ultrasonic transducer imparts ultrasonic energy to the cleaning fluid, facilitating the removal of contaminants from both the interior and exterior of the instruments being processed. A removable cover is provided over the top of the tub to contain over-spray from the cleaning process, and to protect the surgical instruments being processed from outside contamination.

The cleaning process involves, in general terms, the loading of instruments to be cleaned in a tray, and connection of the instruments to be cleaned to a source of cleaning solution supplied through a manifold. The instruments to be cleaned are fully immersed in a cleaning solution, while at the same time the interior of the instruments to be cleaned is provided with a flow of the same cleaning solution to the interior of such instruments. While the instruments are still immersed and supplied, the cleaning solution is energized by ultrasonic waves generated by an ultrasonic transducer, and the solution provides the necessary scrubbing action to dislodge contaminants from the interior and exterior of the instrument. The cleaning process is followed by a flushing process, and the entire cleaning process is repeated multiple times. Between cleaning cycles, the instruments are flushed with pressurized air and water. At the end of the cleaning process, a disinfecting process takes place through the use of high temperature water, and a rinse with water infused with ozone.

### DETAILED DESCRIPTION OF THE DRAWINGS

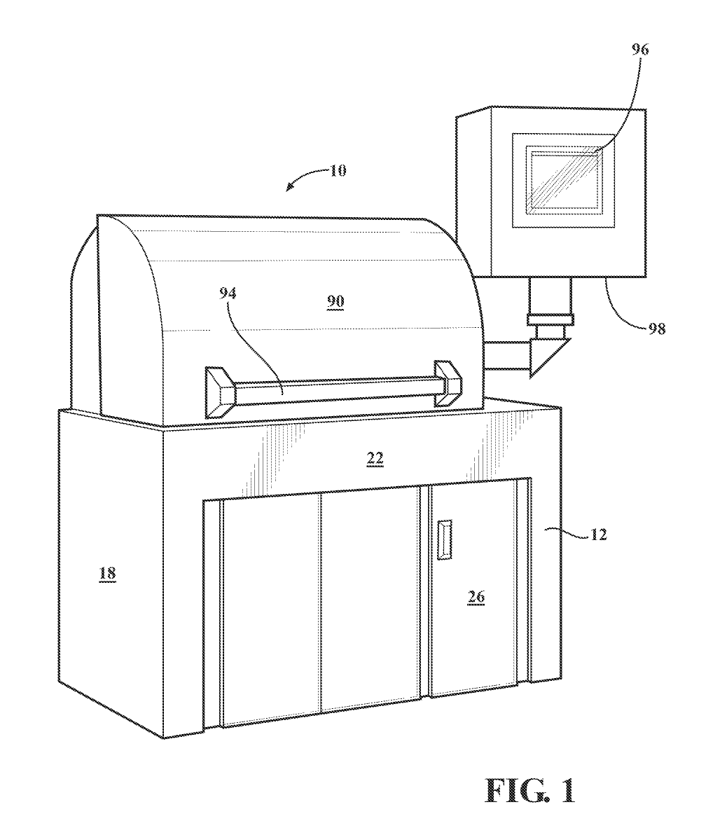

FIG. 1 is a perspective view of the exterior of the apparatus of the present invention.

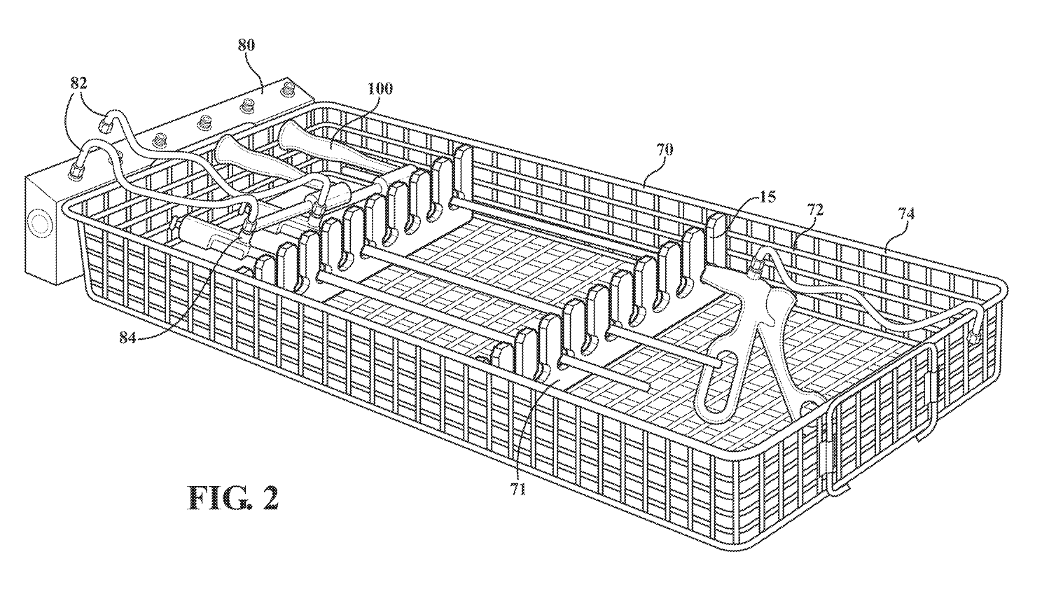

FIG. 2 is a perspective view of a rack holding laparoscopic instruments as used in the invention.

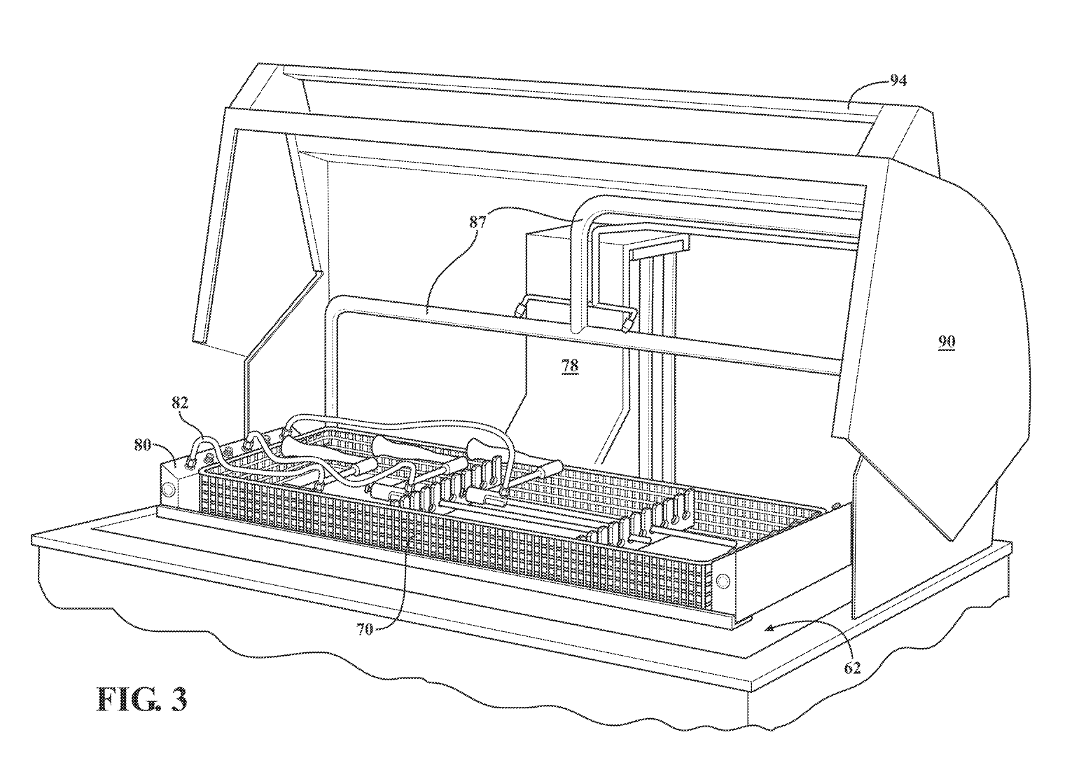

FIG. 3 is a perspective view of a portion of an embodiment of the present invention showing a rack mounted therein.

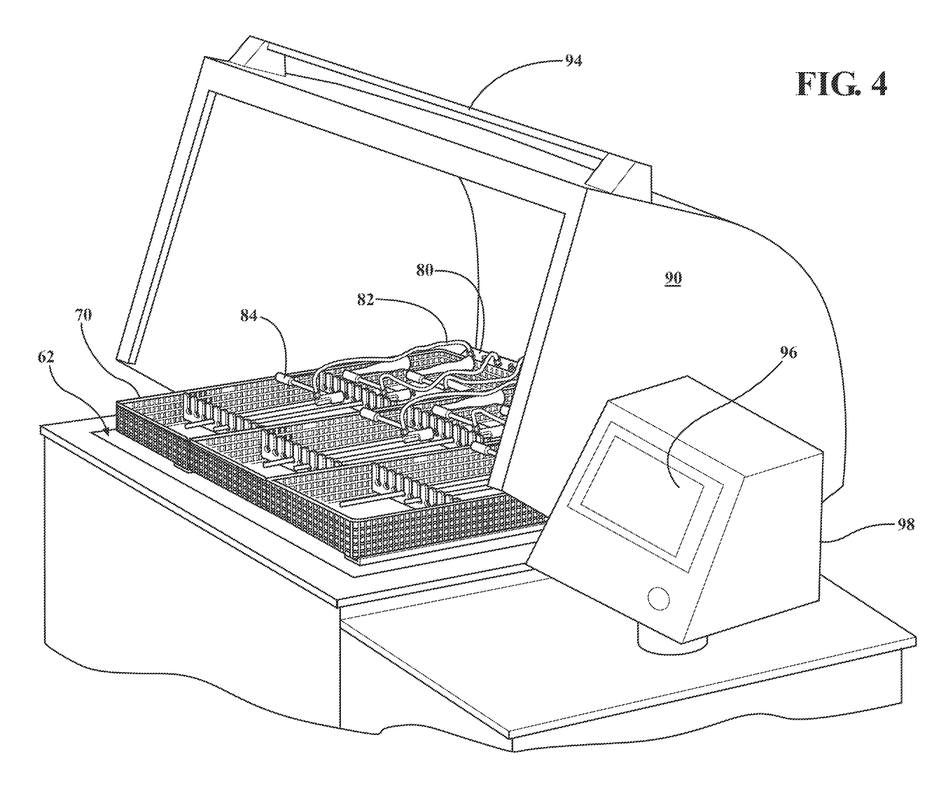

FIG. 4 is a perspective view of an embodiment of the present invention showing three loaded racks mounted therein.

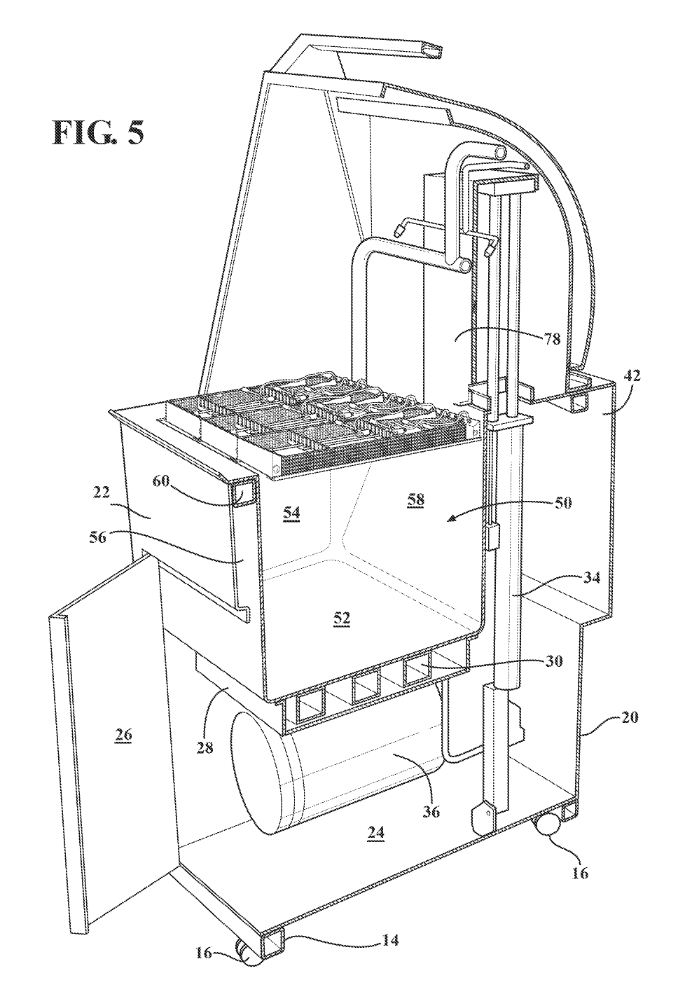

FIG. 5 is a cutaway perspective view of a portion of an embodiment of the present invention.

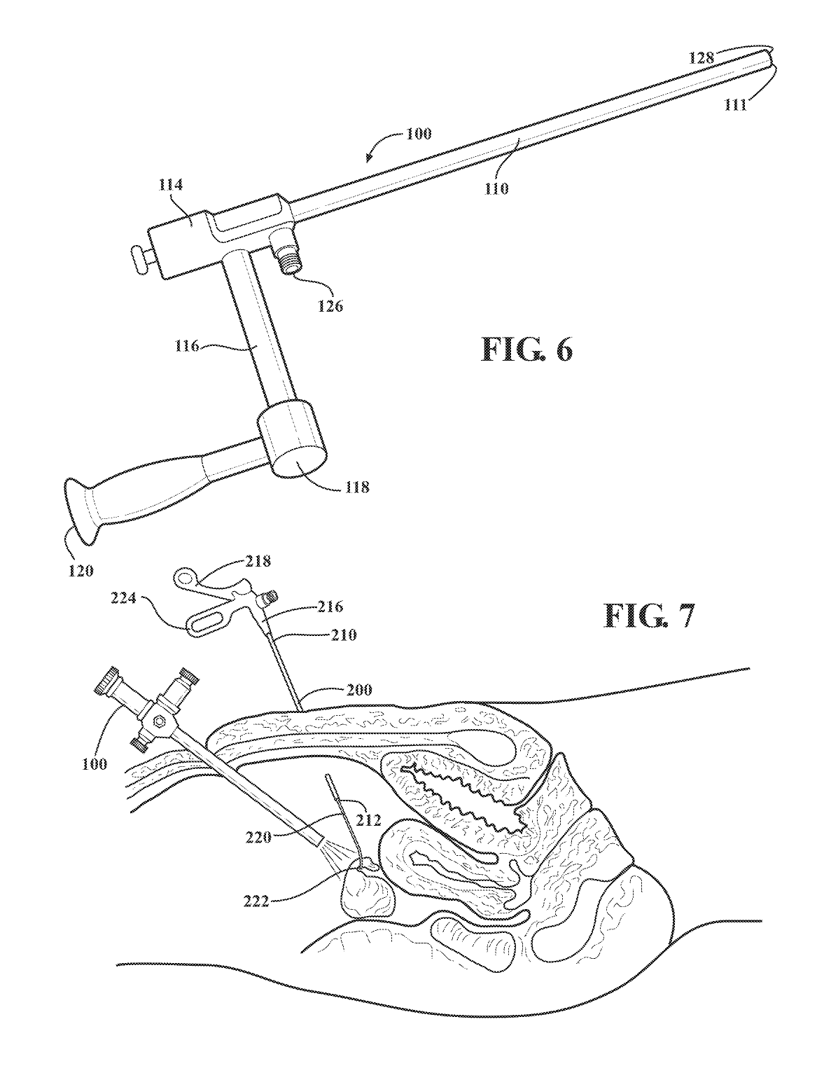

FIG. 6 is a perspective view of a typical laparoscopic instrument.

FIG. 7 is a stylized view of the use of laparoscopic instruments in surgery.

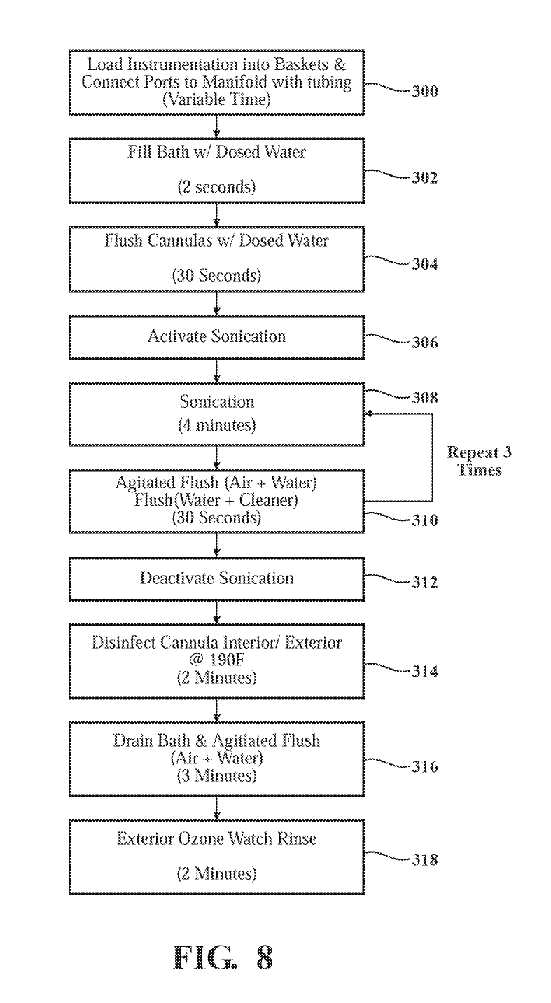

FIG. 8 is a flowchart showing the steps of the inventive process.

### DETAILED DESCRIPTION OF THE EMBODIMENT

The invention will be best understood by a thorough study of the above-referenced drawings, while at the same time referring to the description which follows: As shown in FIG. 6 and FIG. 7, a typical laparoscope 100 comprises an elongated barrel 110 having a bore or lumen 111 along its entire length. Typically, these instruments are manufactured from durable, non-oxidizing metals such as stainless steel. The length of a typical laparoscope is 6-12 inches, with precise measurements adapted for the type of surgical application in which the laparoscope will be used. The barrel 110 is secured to a body 114, to which is attached a grip 116. The body 114 and grip 116 are connected to an optical chamber 118 which may include one or more mirrors. Connected to the grip with an optical chamber 118 is an eye piece 120. The laparoscope 100 may incorporate an integral light source, or be adapted to engage with an external light source, which may be in the form of a flexible optical fiber bundle affixed to a light source input secured to a portion of the optical chamber 118. At the distal end of the barrel 110 are positioned one or more optical lenses (not shown), as well as an outlet for light transmitted by the light source. The outlet may be in the form of a lens, a diffusion grating or window suitable for allowing transmission of light from the light source through the lumen and into the body cavity which is the subject of the surgical procedure being performed. To facilitate the introduction of carbon dioxide or other gases into the body cavity which is the subject of the surgical procedure, a laparoscope is typically provided with an inlet 126 and an outlet 128. Preferably, the optical elements of the laparoscope are sealed to prevent the ingress of bio-contaminants into the lumen of the laparoscope. By supplying pressurized gas, such as carbon dioxide, to the inlet 126, corresponding gas transmission takes place through the outlet 128, thereby introducing the gas into the body cavity. Typically, the laparoscope's barrel is inserted into the patient's body through a small incision between 2 millimeters and 5 millimeters in length.

A typical surgical operation using laparoscopic instruments is depicted in FIG. 7. These instruments may include a cutter of the scissor type, containing a pair of opposed blades which are pivotally interconnected to facilitate a scissor cut. In this type of laparoscopic surgical instrument 200, there is a hollow barrel 210 having an inner lumen 212. The barrel 210 is attached to a receiver 216 which is provided with a grip 218 and a trigger 224, pivotally interconnected with the grip 218. An actuator rod 220 extends from the trigger 224 to the operative element 222 of the instrument 200. In this fashion, operation of the trigger causes a translation of the actuator rod 220 within the lumen 212 of the barrel 210, and operates one or both of the blades at the distal end of the barrel 210. Preferably, the coaxial fit of the actuator rod 220 within the barrel 210 is precise, and may include one or more seals (not shown) between the actuator rod 220 and the lumen 212 to minimize the ingress of contaminants from the patient's body cavity into the lumen 212 of the laparoscopic surgical instrument 200.

A typical use of the laparoscope and a laparoscopic surgical instrument is also depicted in FIG. 7. Here, a laparoscope 100 is inserted through an incision in a patient's abdomen, and gas has been injected into the cavity to provide the surgeon a visual surgical field for the contents of the abdominal cavity being explored. At the same time, a laparoscopic surgical instrument 200 is inserted into the abdominal cavity through a second incision. While the laparoscope provides a source for maintaining gas pressure within the abdominal cavity, and provides the surgeon a view of the surgical field, the actual surgical procedures are performed using one or more laparoscopic instruments 200. While observing the surgical field through the laparoscope, the surgeon may operate one or more laparoscopic instruments to hold, cut, staple, etc., the various tissues which are manipulated during a typical laparoscopic surgical procedure.

It will be appreciated that the same type of methodologies are used in other surgeries, such as thorascopic surgeries. Although the laparoscopes and laparoscopic surgical instruments are provided with sealing means to prevent the ingress to the interior of the instrument of contaminants in the form of body fluid or minute pieces of tissue, it is an unfortunate reality that such ingress does and will occur. No seal is immune from leakage, and it happens that, in a normal surgical procedure, a variety of bio-mass materials finds its way into the lumen of laparoscopes and laparoscopic surgical instruments. Because the design of these devices limits the ability to disassemble them for cleaning, it is desirable to have effective machines and methodologies for removing these contaminants from the laparoscopic devices to minimize the spread of infection and to insure the continued functionality of the instruments themselves.

The invention is a method and apparatus for accomplishing these goals. Laparoscopes and laparoscopic instruments are provided with nipples or similar fittings at inlets 126 to facilitate the cleaning process. In the present embodiment, a surgical instrument is provided with an inlet 126 to which a length of tubing may be removably attached utilizing a clamp, or similar clamping device to insure an airtight and watertight seal between a fluid source and the lumen of the instrument. During the cleaning process, a fluid source provides the necessary flow of fluid used for cleaning and disinfecting. In typical applications, the fluid used is water containing a dissolved enzymatic cleaning agent, although other cleaning fluids of various descriptions may also be used. The fluid source communicates with a fluid heater, so that fluid from the fluid source is introduced into the fluid heater prior to being presented to the instrument for cleaning. In one embodiment, the fluid heater elevates the temperature of the fluid to approximately 120° F., and fluid flowing to the invention may be maintained at that temperature through the use of a fluid thermostat associated with the fluid heater. Typically, such a thermostat is controllable, so that the precise temperature of the cleaning fluid being presented to the laparoscopic instrument can be controlled. Additionally, it is desirable that the fluid be introduced into the interior of the laparoscopic surgical instrument under a predetermined amount of pressure. Typical pressures are between 10 and 50 psi. To insure that the desired amount of pressure is maintained, a pressure regulator may be connected between the fluid source and the laparoscopic instrument during the cleaning process.

While hot pressurized cleaning fluid is useful in the cleaning process, it is known that such a configuration will not, by itself, produce the best possible levels of cleanliness. Accordingly, the invention further comprises a bubble generating device which introduces microscopic bubbles of approximately 15 microns in diameter, into the fluid stream being presented to the instrument being cleaned. In the present device, fluid passes into a bubble generator having a substantially cylindrical inner passageway and one or more inlets for gas, said inlets communicating with said passageway. As the gas enters the passageway, it is intermixed with the water, producing extremely small diameter bubbles. Since the surface tension of the air bubble in water presents stronger impact forces, the bubbles will collide with the contaminants found in the lumen of the instrument, and, together with the force of the water under pressure creates a stress break in the binding of the bio-mass to the surface of the tool, dislodging the bio-mass and suspending it in the fluid.

Additional cleaning effectiveness is obtained through the use of an ultrasonic wave generator co-located in the fluid path. The ultrasonic generator, using an oscillating element introduces ultrasonic wave frequencies into the fluid/bubble stream, causing the fluid and bubbles to oscillate in opposing directions. As the fluid and the dispersed bubbles move rapidly back and forth under the influence of the ultrasonic oscillator, the fluid and bubbles strike and dislodge contaminants contained within the lumen 111 of the laparoscope 100.

To avoid dead or null spots within the lumen resulting from standing waves being created by the ultrasonic frequencies, the ultrasonic generator is designed to “sweep” across a range of frequencies during the cleaning cycle, thereby insuring that no standing waves are created, and further insuring that effective cleaning mechanical forces are created on all surfaces of the lumen of the instrument. The ultrasonic frequency controller is preferably automatic, operating in the frequency range from 25 KHZ to 150 KHZ on a predetermined cycle. However, it is also contemplated within the present invention that the ultrasonic generator may be manually controlled as well.

The typical cleaning and disinfecting cycle for a single instrument requires approximately 30 minutes of processing utilizing high temperature fluid at approximately 50 PSI with ultrasonic scrubbing taking place as above described. Accordingly, to make the most efficient use of the apparatus and methodology described, it is desirable to have a manifold 80 connected to the fluid source, heater, pressure regulator, ultrasonic generator combination. A plurality of laparoscopes 100 may be secured to a cleaning rack 70 attached to the manifold 80 and simultaneously connected to a single source of fluid. In this fashion, a number of laparoscopes 100 may be simultaneously cleaned in a single timed operation utilizing a common fluid source and common fluid conditioning as above-described.

As the cleaning process progresses, liquid or liquefied contaminants are dissolved or suspended in the cleaning fluid, where they will remain in suspension for sufficient period of time to allow the cleaning fluid to be extracted or drained from the instrument, thereby carrying with it the contaminants so removed. If necessary, the instruments may be provided with a fluid outlet to allow a continuous cycling of fluids through the instrument. In another embodiment, the fluids introduced into the lumen of the laparoscopic instrument may be suctioned from the instrument, and the process repeated as many times as necessary until the cleaning fluids demonstrate no remaining traces of contaminants.

In the preferred embodiment, the methodology for the entire cleaning process is set forth in the process flow diagram of FIG. 8 and depicted in FIGS. 1-6. The method begins with the placement of the laparoscopes 100 to be cleaned within the cleaning enclosure, where each such laparoscope 100 is secured in a cleaning rack 70. Each inlet 126 is connected by a supply tube 82 to a manifold 80 with a quick disconnect connector 84. The cleaning rack 70 is attached to the manifold 80, so that a laparoscope 100 may be inserted into the cleaning rack 70 and connected to the manifold 80 via supply tube 82 at a location separate from the cleaning station 10, which may be more convenient for the operator. The manifold 80 is, in turn, connected to a cleaning fluid source, which is typically a conduit or vessel containing water heated to a predetermined temperature, in which are dissolved a detergent and an enzymatic cleaner. This fluid is then injected into the inlets of the laparoscopes 100 through the use of a pump or other pressurizing source which forces the fluid from the vessel into the manifold 80 and into the inlets 126, where it enters the lumen 111 of the laparoscope100 and flows out of the outlet 128. This initial process serves to initially flush the lumens 111 of the instruments 100, thereby flushing out a portion of the contaminants contained therein, while at the same time filling the lumens 111 with the heated solution.

In the next step in the inventive process, an ultrasonic generator 119 is utilized to impart high frequency energy into the solution. Typically, the ultrasonic frequency generated is in the frequency range of 25 k to 150 k, and the generator “sweeps” across this frequency, imparting ultrasonic pulses into the solution across the frequency band so selected. The ultrasonic impulses are transmitted to the solution, which then further dislodges any contaminants contained within the lumen 111. The inventive method then involves the application of similar ultrasonic energy to the solution passing through the lumen for a period of approximately 4 minutes. Thereafter, air and water heated to at least 150° F. is introduced into the lumen 111, again utilizing the ultrasonic frequency controller for a period of approximately 15 seconds. The lumen 111 is then flushed with a solution of water and enzymatic cleaner for approximately 15 seconds. This cycle of sonication, agitation with aerated water and flushing is repeated three times. Throughout the process, the trays in which the instruments are held may also be agitated.

In the next step, the laparoscopic instrument 100 is heated, utilizing hot water, to a temperature of at least 190° F. for disinfection purposes for about two minutes. Thereafter, the lumen 111 is flushed with aerated water for about three minutes and drained, and the discharged solution from the cleaning process is flushed from the tub 50 and disposed of as waste, and finally, the instrument exterior is rinsed with water infused with ozone, called ozonated water, for a period of about 2 minutes to complete the disinfection process.

With reference now to FIGS. 1-5, a cleaning station 10 according to the present invention is depicted, and its components will be explained in detail. The cleaning station is essentially self-contained, containing all the necessary electrical, fluid-handling and ultrasonic energy components to affect a thorough cleaning and disinfection of laparoscopic surgical instruments 100. In the preferred embodiment, the cleaning station 10 is provided with a plurality of mounting points 15, so that several laparoscopic instruments 100 may be cleaned and disinfected simultaneously. The cleaning station 10 is a unitary, self-contained apparatus. It requires only a source of fresh water, compressed air and electricity for operation.

The cleaning station 10 comprises a lower enclosure 12 structurally supported by lower enclosure support frame 14. Preferably, one or more casters 16 are fitted to the lower enclosure support frame 14 to enable the cleaning station to be easily moved between locations without the need for separate transporting apparatus. Additionally, the provision for casters 16 permits the cleaning station 10 to be positioned in close proximity to institutional sources of water, compressed air and electricity. Preferably, the enclosure 12 is fabricated from sturdy material such as stainless steel, which is rugged, durable and easy to clean. The lower enclosure 12 includes sidewalls 18, a rear wall 20, a front wall 22 and a floor 24. Doors 26 are provided to facilitate access to the interior of the lower enclosure 12. The lower enclosure 12 also includes an upper frame 28 and super-structure 30. The upper frame 28 and super-structure 30 elements are necessary to support the weight associated with the remaining portions of the cleaning station 10, which includes a tub 50 having a significant capacity. Since the tub 50 is regularly filled with several gallons of aqueous solution, it is necessary that the lower enclosure 12 and its framework be sturdy enough to support the substantial weight of the cleaning fluid being circulated, stored and utilized by the apparatus. Mounted partially within the lower enclosure 12 is a pneumatic actuator 34, a cleaning fluid supply vessel (not shown), an ultrasonic transducer 36, and a number of valves and pumps for regulation of the flow of cleaning fluid, air and water within and to and from the cleaning station.

Mounted to the lower enclosure 12 is an upper enclosure 42 which houses and protects remaining elements of the cleaning station 10. The upper enclosure is anterior to the tub 50. Tub 50 is formed with side walls 54, tub bottom 52, tub front 56 and tub back 58, so as to form a five-sided enclosure open at the top. Reinforcements 60 surround the perimeter of tub 50 to provide the necessary structural support for the tub 50, which is designed to carry the weight of a substantial volume of aqueous solution. The bottom, side walls, front, and back of the tub form a tub interior, and the tub bottom 52 is provided with one or more controllable drains (not shown) to facilitate removal of the aqueous cleaning fluid used by the system.

In the embodiment, a plurality of cleaning racks 70 formed from a rack framework 74 and screen material 72 are provided to hold one or more laparoscopes 100 during the cleaning and disinfecting process. Typically, each rack is provided with instrument-support elements 71 to align and support the instruments to be cleaned within the confines of the walls and floor of the cleaning racks 70. Attached to the end of each rack is a manifold 80. Each manifold is provided with a plurality of supply tubes 82 at an end proximal to the manifold 80, the supply tubes 82 being provided with quick disconnect connector 84 at the distal ends of the supply tubes. Each manifold 80 is provided with a fluid supply inlet. Fluid supply pipes 87 serve to route fluid and air to the manifolds 80 under automated control facilitated by a plurality of valves. The tub 50 is further provided with a hood 90 mounted to hood pivots, and provided with a hood handle 94. The hood 90 may be rotated to the open position to facilitate loading of the cleaning racks 70 to the moveable backplane 78 and, in general, to provide access to the interior of the cleaning station 10. In a closed position, the hood 90 serves to contain agitated fluids within the tub and hood interior, and to protect the laparoscopes 100 during the cleaning process from unwanted contamination.

The cleaning station 10 thus described contains all the elements necessary for supplying heated water, agitated aqueous solution, and electrical power during the cleaning process. It is contemplated that the cleaning process takes place under automated control through the use of a program logic controller 98, the status and performance of which is displayed on a monitor 96. The program logic controller 98 can be implemented using a computer or other computing device. In the preferred embodiment, the monitor 96 constitutes a touch-sensitive display screen, having a graphical user interface which presents to the user a series of options for controlling the apparatus, and provides the status of the operation of the cleaning and disinfecting cycle to an operator. The controller is capable of network connectivity, so that the status of the cleaning and disinfecting operation can be reported to locations remote from the cleaning station 10 over local and wide area networks. In addition, wireless personal area and local area network connections may be made utilizing the controller, allowing scheduled and/or remote operation of the cleaning process both locally, and from locations remote from the physical location of the cleaning station 10.

In operation, one or more surgical laparoscopes 100 are placed in one or more cleaning racks 70 provided with instrument-support elements 71 and a manifold 80. As shown in FIG. 6, each such laparoscope 100 is typically provided with an inlet 126 and outlet 128 designed to facilitate the passage through the laparoscope 100 of a cleaning medium. As the laparoscopes 100 are placed into the cleaning racks 70, supply tubes 82 from manifold 80 are connected using quick disconnect connector 84 to each of the laparoscopes 100 mounted in the cleaning racks 70 via inlet 126.

The cleaning process can be further understood with reference to the flowchart of FIG. 8. In the first step 300, cleaning racks 70 are loaded with laparoscopes 100 and attached to manifolds 80 with supply tubing 82 using quick disconnect connectors 84. The manifolds 80 are then attached to a movable backplane 78 which is movably secured to a pneumatic actuator 34 supported by the lower enclosure 12 of the cleaning station 10. The manifolds 80 may be mounted to the cleaning racks 70, so that the manifolds 80 and cleaning racks 70 are removable, as a unit, from the movable backplane 78. In this fashion, the interconnections between the manifolds 80 and laparoscopes 100 may be made while the cleaning racks 70 are outside the cleaning station 10, and a single fluid connection may be made to the manifold 80 from the cleaning station 10. Once the racks have been inserted in the tub interior 62 by attachment to movable backplane 78, a fluid connection exists between the cleaning fluid source provided by the cleaning station 10 and the laparoscopes 100 to be cleaned and disinfected.

Upon completion of the positioning of the racks on the movable backplane 78, in step 302 pneumatic actuator 34 lowers the movable backplane 78 and the associated cleaning racks 70 into the tub interior 62. Heated water is then allowed to flow into the water supply inlet of the cleaning station 10, through a venturi assembly. The venturi assembly contains a third fitting which is attached to a tube, the distal end of which is connected to a supply of enzymatic cleaner. Water flowing through the venturi draws a metered amount of enzymatic cleaner into the stream of water flowing into the cleaning station 10. This initial flow of cleaning fluid is utilized to fill the interior of tub 50 so that the cleaning racks 70 and the laparoscopes 100 which they contain are completed immersed. Once the tub 50 is substantially filled, in step 304 control valves redirect the cleaning fluid to the manifolds 80, and the lumens 111 of the laparoscopes 100 being cleaned are accordingly flushed and filled with cleaning fluid. Once the tub 50 has been filled and cleaning fluid has been infused into the lumens 111 of the laparoscopes 100, the initial sonication step 306 takes place.

Sonication is initiated at step 306 and continues until sonic processing is complete in step 312. The initial sonication step 308 continues for approximately four minutes. It has been learned that sonication for two minutes or more is sufficient to remove substantially microscopic air bubbles from the lumens 111 of the laparoscopes 100. Thus the first two minutes of the first four minute sonication will serve to remove unwanted air bubbles. During this sonication process, ultrasonic energy is imparted to the cleaning fluid by the ultrasonic transducer over a varying range of frequencies, typically between 25k and 150k. The frequency so generated by the ultrasonic transducer may sweep across this entire frequency range. After approximately four minutes, fresh water and compressed air are introduced into the manifold 80 utilizing control valves in step 310. During this flushing cycle, sonication continues to occur. After approximately fifteen seconds of combined flushing with air and water and sonication, the lumens 111 of the instruments 100 are then re-injected with cleaning fluid, which may take about 15 seconds. Following this, the routine loops back to step 308 and an additional four minutes of sonication begins. The cycle of sonication, agitated flush using air and water, and flushing using cleaning fluid is repeated two additional times, for a total of three cycles.

Following this, in step 312 the sonication is deactivated. Next, in step 314, water heated to a temperature of 190° is injected into the manifolds 80, through supply tubes 82, and into the lumens 111 of the laparoscopes 100 for a period of two minutes, thereby disinfecting the interior of the laparoscopes 100. Following this disinfection step, in step 316 the tub 50 is drained, while at the same time air and water are infused into the manifolds 80, through supply tubes 82 and into the lumens 111 of the laparoscopes 100. In the final cleaning step 318 the exterior of the laparoscopes 100 is rinsed in water infused with ozone for a period of two minutes, affecting disinfection of the exterior of the laparoscopes 100. Once the cleaning cycle has been completed, a report of the process steps is stored within the program logic controller 98 and may be displayed on the monitor 96 or may optionally be sent to a remote location for viewing or printing.

Throughout the cleaning and disinfecting cycle above described, it is desirable to agitate the laparoscopes 100, by agitation of the cleaning racks 70 in which the laparoscopes 100 are mounted. This step may be accomplished by operation of the pneumatic actuator 34 to which the movable backplane 78 is secured. In this fashion, the cleaning racks 70 may be vertically oscillated within the cleaning fluid contained within the tub 50 while the flushing and sonication processes are underway.

The above-described embodiments have been described in order to allow easy understanding of the present invention and do not limit the present invention. On the contrary, the invention is intended to cover various modification and equivalent arrangements included within the scope of the appended claims, which scope is to be accorded the broadest interpretations so as to encompass all such modifications and equivalent structures is permitted under the law.

I claim:

1. A method for removing contaminants from laparoscopic devices comprising:
A. providing a cleaning station having:
heated cleaning fluid;
a source of gas to be inter-mixed with said fluid;
a source of ozone and water; and,
a source of ultrasonic energy to be associated with said fluid and gas;
B. connecting said laparoscopic devices to said cleaning station to permit said fluid and gas to be introduced into the interior of said laparoscopic devices;
C. immersing said laparoscopic devices in said fluid and gas in said cleaning station and flushing said laparoscopic devices with said fluid and gas;
D. activating said ultrasonic energy
E. sonically cleaning said laparoscopic devices for about four minutes followed by flushing said laparoscopic devices for about 30 seconds;
F. deactivating said ultrasonic energy;
G. disinfecting said laparoscopic devices with hot fluid, draining said fluid and gas from said cleaning station and further disinfecting said laparoscopic devices with an ozone and water rinse.

2. The method of claim 1 wherein the heated cleaning fluid comprises an enzymatic, pH neutral cleaning agent.

3. The method of claim 1 wherein the gas is room air.

4. The method of claim 1 wherein the ultrasonic energy found in the frequencies between 25 kHz and 150 kHz.

5. The method of claim 1 wherein the step of sonically cleaning said laparoscopic devices followed by flushing said laparoscopic instruments is repeated three times.

6. The method of claim 1 wherein said disinfecting said laparoscopic devices further comprises flushing the interior and exterior of said laparoscopic devices with 190° F. fluid for about two minutes.

7. The method of claim 1 wherein said ozone and water rinse is performed with ozonated water for about two minutes.

8. An apparatus for removing contaminants from laparoscopic devices comprising:
a source of heated cleaning fluid;
a source of gas to be inter-mixed with said fluid;
a source of ozone;
a source of water;
a source of ultrasonic energy to be associated with said fluid and said gas;
a basket for holding said laparoscopic devices and connecting them to said source of heated cleaning fluid;
a tub for immersing said laparoscopic devices in said heated cleaning fluid;
a pneumatic actuator for lowering said basket holding said laparoscopic devices into said tub and agitating said laparoscopic devices; and
a controller for controlling said source of heated cleaning fluid, said source of gas, said source of ozone, said source of water, said source of ultrasonic energy and said pneumatic actuator.

9. The apparatus of claim 8 wherein said basket for holding said laparoscopic devices includes a manifold having at least one connection for introducing said cleaning fluid into said laparoscopic devices and a single connection for connecting said manifold to said source of heated cleaning fluid.
